(19) United States
(12) Patent Application Publication
(10) Pub. No.: US 2006/0198768 A1
(43) Pub. Date: Sep. 7, 2006

(54) Diffuser

(76) Inventors: Anil J. Gupte, St. Charles, IL (US); Juan Vasquez, Bartlett, IL (US); Lanee Reuther, Naperville, IL (US); Angela M. Clark, Cambridge, MA (US); Laney Pellegrini, Morris, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/074,276

(22) Filed: Mar. 7, 2005

(51) Int. Cl. A61L 9/03

(52) U.S. Cl. 422/125

(57) ABSTRACT

A diffuser is provided. The diffuser includes a frame, a heat source associated with the frame, and a plurality of strips of heat-responsive material attached to the frame, each strip of heat-responsive material comprising a carrier and an active component, the active component comprising at least one of a fragrance, an insecticide, or a medicinal preparation.

### BACKGROUND

This patent is directed to a diffuser, and in particular to a diffuser that may utilize a heat source to cause a heat-responsive material to release an active component into the environment.

### SUMMARY OF THE INVENTION

In one aspect, a diffuser is provided. The diffuser includes a frame, a heat source associated with the frame, and a plurality of strips of heat-responsive material attached to the frame, each strip of heat-responsive material comprising a carrier and an active component, the active component comprising at least one of a fragrance, an insecticide, or a medicinal preparation.

In another aspect, another diffuser is provided. The diffuser includes a frame including a receptacle, a candle disposed in the receptacle, and a plurality of refills attached to the frame and disposed about the receptacle, each of the plurality of refills including a layer of heat-responsive material, the heat-responsive material comprising a carrier and an active component, the active component comprising at least one of a fragrance, an insecticide, or a medicinal preparation.

Additional aspects of the disclosure are defined by the claims of this patent.

### BRIEF DESCRIPTION OF THE DRAWINGS

FIG. 1 is a perspective view of an embodiment of a diffuser according to the present disclosure;

FIG. 2 is a perspective view of the diffuser of FIG. 1 with a refill partially removed from a slot;

FIG. 3 is a perspective view of a second embodiment of a diffuser;

FIG. 4 is a perspective view of a refill used with the diffuser of FIG. 3;

FIG. 5 is a perspective view of a third embodiment of a diffuser;

FIG. 6 is an exploded view of a fourth embodiment of a diffuser;

FIG. 7 is a perspective view of a fifth embodiment of a diffuser; and

FIG. 8 is a perspective view of a sixth embodiment of a diffuser.

### DETAILED DESCRIPTION OF VARIOUS EMBODIMENTS

Although the following text sets forth a detailed description of numerous different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

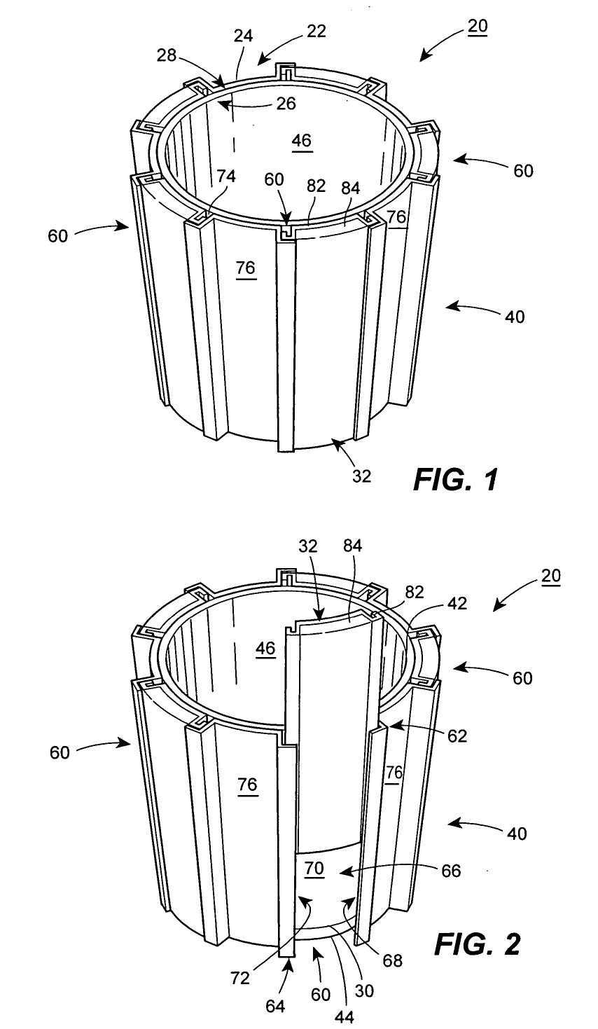

FIGS. 1 and 2 illustrate an embodiment of a diffuser 20. In general terms, the diffuser 20 may include a frame 22 having a wall 24 with first and second sides 26, 28. A heat source 30 may be disposed on the first side 26 of the frame wall 24, and a layer 32 of heat-responsive material may be disposed on the second side 28 of the frame wall 24

The layer 32 of heat-responsive material may include a carrier and an active component. The active component may be a fragrance, an insecticide, or a medicinal preparation, or a combination of any two or more of these components. Furthermore, other active components may be utilized as well. The layer 32 may be heat-responsive in that the layer 32 may release the active component when heat is transferred to the layer 32, although it may also be the case that the layer 32 may release the active component even under conditions where the heat source 30 is not transferring heat to the layer 32.

In operation, the heat source 30 may release heat, which heat may be absorbed by the layer 32. In fact, after the heat source 30 releases the heat, the heat may be conducted from the first side 26 to the second side 28 of the wall 24, and from the second side 28 to the layer 32. With the addition of heat to the layer 32, the active component, whether that be a fragrance, an insecticide, a medicinal preparation or a combination of any two or more these components or other chemical composition, may be released into the environment.

More specifically, returning to FIG. 1, the frame wall 24 may define a cylindrically-shaped holder 40. In particular, the frame wall 24 may include a cylindrically-shaped side wall 42, which may taper slightly, and a circular bottom wall 44. The side wall 42 and the bottom wall 44 may be of a one-piece construction, also as shown, although this need not be the case. For example, the side and bottom walls 42, 44 may be formed separately and then attached or secured to each other, or the bottom wall 44 may be omitted altogether. As shown, however, the side wall 42 and the bottom wall 44 may collectively define a receptacle 46.

While a substantially cylindrically-shaped holder 40 is shown, it will be recognized that other geometries may be employed. That is, instead of a circular bottom wall 44, a square-shaped bottom wall may be used. Moreover, rather than an annular, cylindrically-shaped side wall 42, a plurality of side walls, which may be square-shape as well, may be used. The overall appearance of the frame 22 may thus be cubic instead of cylindrical or conic. As still other examples, the frame 22 may have a spherical or pyramid shape. Moreover, the geometry used need not be symmetrical about multiple axes, as shown; in fact, it need not be symmetrical about any axis.

The heat source 30 may be disposed within the receptacle 46 and may include a candle. As shown, the candle may be shorter than the height of the wall 42, although the diffuser 20 may function adequately if the candle is the same height as or taller than the height of the wall 42. Other heat sources may also be used, as explained in greater detail below relative to the other illustrated embodiments.

Also according to this embodiment, the second side 28 of the wall 24 may have a plurality of slots 60 thereon. The slots 60 shown in FIG. 1 may have a first open end 62, a second open end 64, and an open side 66, as well as three closed sides 68, 70, 72. The slots 60 may be defined by a surface 74 of the holder wall 24 and a plurality of brackets 76 attached to or secured to the holder wall 24. More specifically, adjacent brackets 76 may be arranged about the open side 66 so as to define the open side 66.

In each of the slots 60 may be disposed a refill 80. The refill 80 may include a U-shaped frame 82 and a strip 84 defined by the layer 32 of heat-responsive material. The refill 80 may be removeably disposed in the slot 60 between a first position wherein the refill 80 is substantially disposed within the slot 60 (FIG. 1) and a second position wherein the refill 80 is spaced from the slot 60 (FIG. 2 shows an intermediate position between the two positions). The refill 80, and in particular the refill frame 82, may cooperate with the adjacent brackets 76 to limit removal of the refill through the open side 66 of the slot 60.

As stated previously, the layer 32 of heat-responsive material forming the strip 84 may include a carrier. The carrier may be selected from materials such as waxes, gels, polymers, and ceramics. The non-exhaustive list of waxes may include waxes from natural, synthetic or petroleum sources. The non-exhaustive list of gels may include gels using hydrocarbon oil, vegetable oil, mineral oil, polyamides, polymers, glycols or glycerols. The non-exhaustive list of polymers may include polyethylene, polypropylene, polyvinyl chloride, polyethylene terephthalate, polystyrene, polycarbonate, ABS, EVA, PEBAX and polymethylpentene. Alternatively, materials such as wood, paper, paperboard and silicon may be used. Preferably, polyamides may be used as the carrier.

The layer 32 of the heat-responsive material forming the strip 84 may also include an active component. The active component may be a fragrance, an insecticide, a medicinal preparation or a combination of any two or more of these components. The fragrance may be selected from the non-exhaustive list of fragrances including musk oil, civet, castreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, cauout oil, salvia oil, clove oil, chamomile oil, costus oil, labdanum oil, broom extract, carrot seed extract, jasmine extract, minmosa extract, narcissus extract, olibanum extract, rose extract, acetophenonene, dimethylinadane derivatives, naphthaline derivatives, allyl caprate, alpha-amylcinnamic aldehyde, anethole, anisaldehyde, benzyl acetate, benzyl alcohol, benzyl propionate, borneol, cinnmayl acetate, cinnamyl alcohol, citral citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl iso-valeratek iso-eugenol, linalol, linalyl acetate, p-methylacetophenone, methyl anthranilate, methyl dihydroasmonate, methyl eugenol, methyl-beta-naphthol ketone, methylphenhlcarbinyl acetate, musk ketol, mustk xylol, 2,5,6-nanodinol, gamma-nanolactone, phenylacetoaldehydodimethyl acetate, beta-phenylethyl alcohol, 3,3,5-trimethylcyclohexanol, gamma-undecalactone, undecenal, vanillin, and mixtures thereof. The insecticide may be selected from the non-exhaustive list of insecticides including citronella, methyl bromide, aluminium phosphide, and magnesium phosphide. The medicinal preparation may be selected from the non-exhaustive list of preparations including eucalyptus oil, menthol, camphor, thymol, turpentine oil, 1-desoxyephedrine, and bornyl acetate. Additionally, other active components may be used.

The layer 32 of heat-responsive material may be formed from the carrier and the active component according to well-known methods. Moreover, the heat-responsive material may be attached to or secured to the frame 82 using known methods of joining materials, such as applying the layer 32 to the frame 82 while the layer 32 is in a liquid or semi-solid form and allowing the layer 32 to stiffen or harden in place. Alternatively, an adhesive may be used to attach the layer 32 to the frame 82.

While all of the refills 80 used with a particular diffuser 20 may include a common active component, a user may use refills 80 in the slots 60 that have different active components. For example, a user may use refills 80 having different, perhaps complementary, fragrances, such as orange and chocolate, in different slots 60. As another alternative, a user may use refills 80 having active components chosen from different categories, such as a fragrance and a medicinal preparation or a fragrance and an insecticide. It will be recognized that this may provide the user with a great degree of freedom of choice in this regard.

Moreover, while the refills 80 as shown include a frame 82 and a strip 84 (defined by the layer 32 of the heat-responsive material), other embodiments of the diffuser may include only the strip 84. That is, the frame wall 24 or the frame wall 24 and brackets 76 may define slots 60 into which the strips 84 may be disposed. Heat released from the heat source 30 may then be absorbed by the strip 84, which strip 84 may decrease in size as the active component is released. When the strip 84 is completely or substantially reduced in size, a new strip 84 may be introduced into the slot 60.

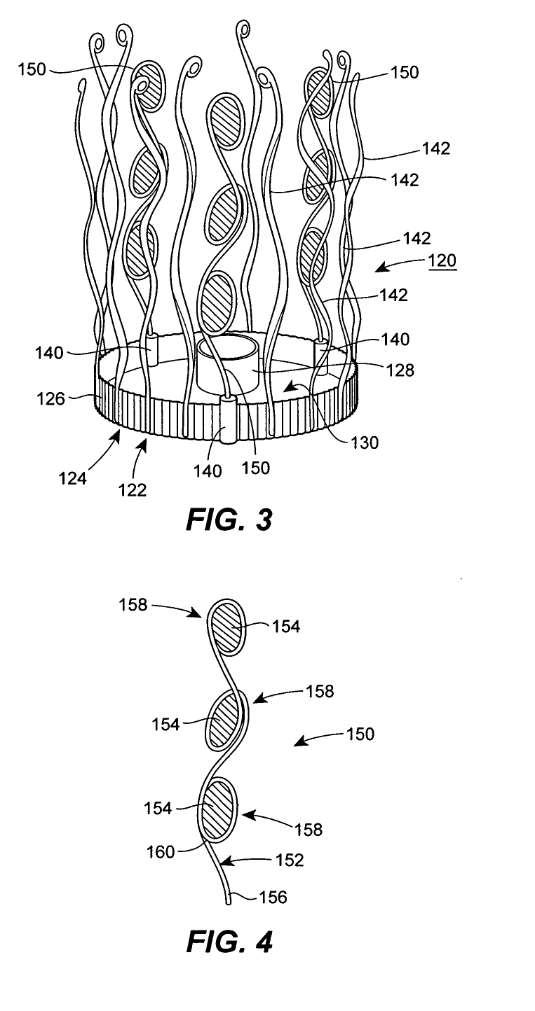

A second embodiment of the diffuser is shown in FIG. 3. The diffuser, designated 120, may include a frame 122. The frame 122 may include a bottom wall 124 and a side wall 126. The bottom wall 124 may be circular in shape, as shown in FIG. 3, although this need not be the case according to all such embodiments. A heat source 128, such as a candle, may be disposed in a receptacle, or holder, 130 defined by the bottom wall 124 and the side wall 126.

The side wall 126 may define or may have attached thereto sockets 140. Each socket 140 may have a circular cross-section as shown, although it is not required that the cross-section be any particular shape. The sockets 140 may be equally spaced about the entire periphery of the bottom wall 124, although the sockets 140 may be disposed in an unequal distribution as well.

Moreover, decorative elements 142 may be disposed between adjacent sockets 140. As shown, five decorative elements 142 are disposed between adjacent sockets 140 with a consistent spacing between the elements 142. However, the decorative elements 142 may be disposed in an unequal distribution (both as to number and spacing) between adjacent sockets 140, may be replaced with additional sockets 140 or may be removed altogether, in which case adjacent sockets 140 may be separated by space alone. While the elements 142 may function as a primarily as decoration, the elements 142 may provide other functionality as well (e.g., as a safety measure to limit access to the heat source 128 disposed in the holder 130).

Disposed in the sockets 140 are refills 150 (see FIG. 4). Each refill 150 may include a frame 152 and at least one strip 154 of heat-responsive material, similar to that referred to above in regard to the strips 84. In particular, the frame 152 may include a thin, wire-like member 156 which has been shaped into one or more loops 158. For example, the wire-like member 156 may be deformed into the loops 158 and then joined to itself, by soldering for example, where the wire-like member 156 crosses itself (such as at a junction 160). The strips 154 may be disposed in the loops 158, for example, by disposing the heat-responsive material in the loops 158 while in a liquid or semi-solid form and then waiting for the heat-responsive material to harden. Alternatively, the strips 154 may be shaped separately, and disposed in the loop 158 by joined the strip 154 to the frame 152 using, for example, an adhesive.

Similar to the diffuser 20 shown in FIGS. 1 and 2, the diffuser 120 includes refills 150 that may be removed from the diffuser 120. In this fashion, the user may include such refills 150 as that user may desire to provide different combinations of fragrances, insectides, and/or medicinal compositions. That is, the user may select certain refills 150 that include heat-responsive material of complementary fragrances. The user may also select a first refill 150 including a strip 154 with a fragrance and a second refill 150 including a strip 154 with an insecticide, so that the fragrance of the first refill 150 modulates the scent of the insecticide of the second refill 150.

Moreover, like the diffuser 20, the diffuser 120 is not limited to the cylindrical geometry shown. Conic, cubic and spherical geometries may also be used. Moreover, the geometry used need not be symmetrical about multiple axes, or even symmetrical about any axis.

Unlike the diffuser 20 shown in FIGS. 1 and 2, the refills 150 are not disposed on an opposite side of a wall relative to the heat source 128. To the contrary, there are no barriers between the heat source 128 and the refills 150, and more specifically the strips 154, in the diffuser 120 shown in FIG. 3. As a consequence, it may be said that the strips 154 define part of an enclosure or wall surrounding the heat source 128. The remainder of the wall may be defined by the decorative elements 142, although as discussed above, the decorative elements 142 may be replaced with refills 150 or removed altogether.

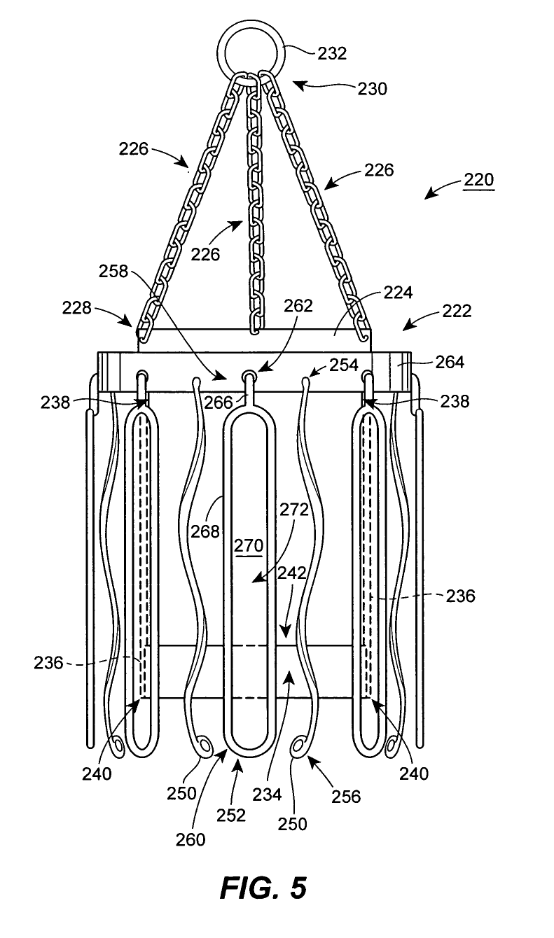

A third embodiment of the diffuser is shown in FIG. 5. The diffuser, designated 220, may include a frame 222. The frame 222 may include a platform 224 to which are attached one or more hangers 226. As illustrated, the hangers 226 may include a number of chains, which chains are joined at first ends 228 to the platform 224 and second ends 230 to a hoop 232, which hoop 232 may in turn may cooperate with a fastener, such as a nail, driven into a window frame, for example, to support the diffuser 220 as shown in FIG. 5.

Disposed beneath the platform 224 may be a heat source holder 234. The holder 234 may be supported by one or more elements attached at their first end to the platform 224 and at their second end to the holder 234. For example, one or more rods 236 may be attached at their first ends 238 to the platform 224 and at their second ends 240 to the holder 234. A heat source 242, such as a candle (e.g., tealight), may be disposed in the holder 234. Additionally, a plurality of decorative elements 250 and refills 252 may be disposed about the holder 234.

The decorative elements 250 may be attached at their first ends 254 to the platform 224 and free at their second ends 256. However, according to certain embodiments, the second ends 256 of some or all of the decorative elements 250 may be attached to the holder 234 to support the holder 234 in conjunction with or in place of the rods 236. The decorative elements 250, as illustrated, may be fixedly secured to the platform 224, although according to other embodiments, the decorative elements 250 may be moveably or removably attached to the platform 224. While the elements 250 may function as a primarily as decoration, the elements 250 may provide other functionality as well (e.g., as a safety measure to limit access to the heat source 242 disposed in the holder 234).

The refills 252 may be attached at their first ends 258 to the platform 224 and free at their second ends 260, similar to the decorative elements 250 shown. Unlike the decorative elements 250, the refills 252 shown are not fixedly secured to the platform 224, although this may be done according to alternative embodiments. Instead, the platform 224 may define a plurality of openings 262 in a wall 264 and the refills 252 may each have a fastener, such as a hook, 266 that may be disposed through one of the openings 262 to removably attach the refill 252 to the platform 224.

The refills 252 may also include a frame 268 attached to the hook 266 and a strip 270. As shown, each frame 268 may define a single oval-shaped loop 272 in which the strip 270 is disposed. The strip 270 may be disposed in the loop 272, for example, by disposing the heat-responsive material in the loop 272 while in a liquid or semi-solid form and then waiting for the heat-responsive material to harden. Alternatively, the strip 270 may be shaped separately, and disposed in the loop 272 by joining the strip 270 to the frame 268 using, for example, an adhesive.

Similar to the diffuser 120 shown in FIGS. 3 and 4, the arrangement of decorative elements 250 and refills 252 shown in FIG. 5 is only one such arrangement. Other arrangements may be possible. For example, while the adjacent refills 252 may be separated by a single decorative element 250 as shown, additional decorative elements 250 may be used, the decorative elements 250 may be replaced with additional refills 252 or the decorative elements 250 removed altogether. Moreover, the spacing of the decorative elements 250 and the refills 252 need not be equal about the entire periphery of the platform 224, as shown. The distribution of decorative elements 240 and/or refills 252 may be unequal in number and spacing about the platform 224.

Moreover, like the diffusers 20, 120, the diffuser 220 is not limited to the cylindrical geometry shown. Conic, cubic and spherical geometries may also be used. Moreover, the geometry used need not be symmetrical about multiple axes, or even symmetrical about any axis.

Similar to the diffusers 20, 120 shown in FIGS. 1-4, the diffuser 220 includes refills 252 that may be removed from the diffuser 220. In this fashion, the user may include such refills 252 as that user may desire to provide different fragrances, insectides, and/or medicinal compositions. That is, the user may select certain refills 252 that include heat-responsive material of complementary fragrances. The user may also select a first refill 252 including a strip 270 with a fragrance and a second refill 252 including a strip 270 with an insecticide, so that the fragrance of the first refill 252 modulates the scent of the insecticide of the second refill 252.

Unlike the diffuser 20 shown in FIGS. 1 and 2 but like the diffuser 120 shown in FIGS. 3 and 4, the refills 252 are not disposed on an opposite side of a wall from the heat source 242. To the contrary, there are no barriers between the heat source 242 and the refills 252, and more specifically the strips 270, in the diffuser 220 shown in FIG. 5. As a consequence, it may be said that the strips 270 define part of an enclosure or wall surrounding the heat source 242. The remainder of the wall may be defined by the decorative elements 250, although as discussed above, the decorative elements 250 may be replaced with refills 252 or removed altogether.

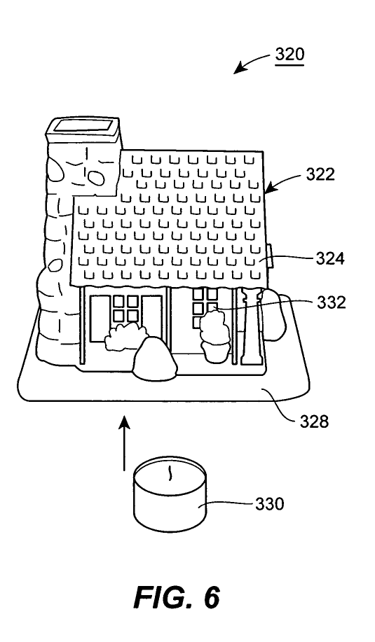

A further embodiment of the diffuser is shown in FIG. 6. The diffuser, designated 320, may include a frame 322 with a wall 324 having a first side (not shown) and a second side 328. The wall 324 may be formed in the shape of a house having at least one window. A candle 330 or other heat source may be disposed inside of a receptacle (not shown) defined by the first side of the wall 324. A layer 332 of heat-responsive material may be disposed on the wall 324. Heat from the lit candle 330 may cause the active component of the heat-responsive material to be released to the environment.

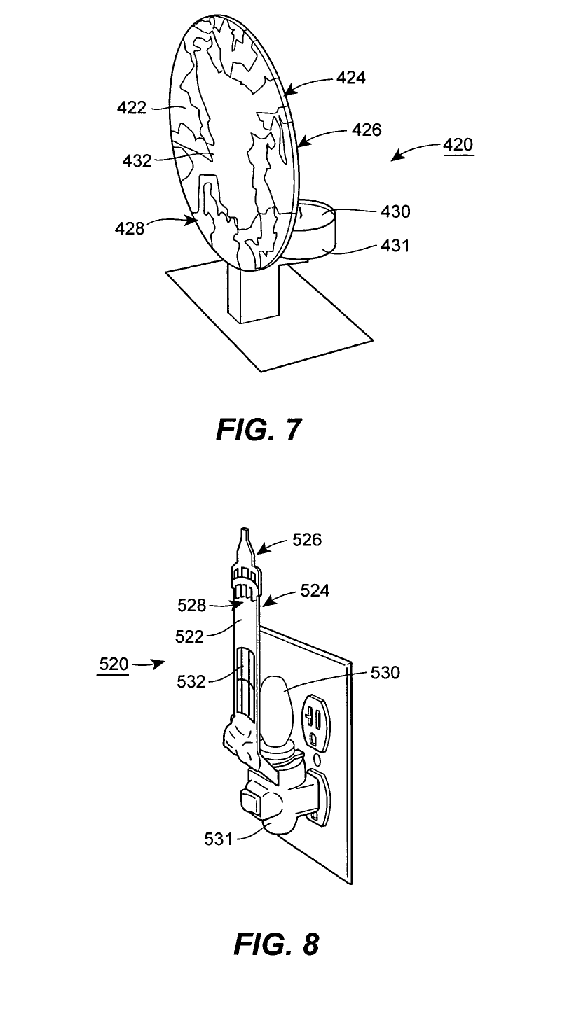

Another alternative embodiment of the diffuser is shown in FIG. 7. The diffuser, designated 420, may include a frame 422 with a wall 424 having a first side 426 and a second side 428. The wall 424 may be formed in the shape of a stained-glass window. A tealight 430 or other heat source may be disposed on the first side 426 of the wall 424, in a holder 431, for example. A layer 432 of heat-responsive material may be disposed on the second side 428 of the wall 424. Heat from the lit candle 430 may cause the active component of the heat-responsive material to be released to the environment.

Yet another alternative embodiment of the diffuser is shown in FIG. 8. The diffuser, designated 530, may include a frame 522 with a wall 524 having a first side 526 and a second side 528. The wall 524 may be formed in the shape of a stained-glass window. A light bulb 530 or other heat source may be disposed on the first side 526 of the wall 524, which light bulb 530 may be coupled to an electrical connector or plug 531 and other circuitry (not shown) which may be required for the operation of the bulb 530. A layer 532 of heat-responsive material may be disposed on the second side 528 of the wall 524. Heat from the light bulb 530 may cause the active component of the heat-responsive material to be released to the environment.

The layers 332, 432, 532 of heat-responsive material used in the diffusers 320, 420, 520 may be removable from the diffusers 320, 420, 520, similar to the strips 84, 154, and 270 in the diffusers 20, 120, 220 discussed above. That is, the layers 332, 432, 532 may be received in recesses or slots formed in the walls 324, 424, 524 of the frames 322, 422, 522. When the layers 332, 432, 532 are used up, the layers 332, 432, 532 may be removed. Alternatively, the layers 332, 432, 532 may be formed of such heat-responsive material that as the active component is used up, the carrier reduces in size as well, until the carrier finally disappears entirely or almost entirely when the active component is entirely or almost entirely diffused.

According to an alternative embodiment, the layers 332, 432, 532 may make up part of the respective walls 324, 424, 524, similar to the strips 154, 270 in the diffusers 120, 220 in FIGS. 3 and 5. According to such an embodiment, the layers 332, 432, 532 may be formed as part of the walls 324, 424, 524. Alternatively, the layers 332, 432, 532 may be disposed into refills, which refills are attached to the walls 324, 424, 524, and may be removed so as to provide the user the ability to selectively dispose refills having different active components or different aspects of a particular active component (e.g., different fragrances).

In addition, the materials which may be used to make any of the embodiments of the diffusers discussed above may be selected from a variety of materials. A non-exhaustive list of such materials may include metals, plastics, resins, glass, fiberglass, ceramics and wood, with the selection of the material selected being recognized by one skilled in the art. A non-exhaustive list of the metals (which may include plated finishes) may include steel (which may be polished, brushed, antiqued, satin finished, embossed, etc.), aluminum (which may be polished, brushed, antiqued, satin finished, galvanized, etc.), iron (which may be wrought, antiqued, etc.), nickel (which may be brushed, antiqued, polished, etc.), brass (which may be polished, antiqued, etc.), gold, silver, pewter, and chrome. A non-exhaustive list of the plastics (which may include copolymers and multi-layer composites) may include acrylonitrile butadiene styrene, high density polyethylene, linear low density polyethylene, low density polyethylene, polyvinyl chloride, polypropylene, polyvinylidiene chloride, polyester teraphthalate, polycarbonate, polystyrene, high impact polystyrene, ethylene ethyl acrylate, ethylene vinyl acetate, polyurethane (which may be cold cast, thermoset, etc.), urea formaldehyde, melamine formaldehyde, phenol formaldehyde and polyesters. A non-exhaustive list of ceramics (which may include glazed and unglazed materials) may include dolomite, stoneware, terra cotta, and porcelain. These materials may be tinted, painted or coated with a variety of materials.

What is claimed is:

1. A diffuser comprising:
a frame;
a heat source associated with the frame;
a plurality of strips of heat-responsive material attached to the frame, each strip of heat-responsive material comprising a carrier and an active component, the active component comprising at least one of a fragrance, an insecticide, or a medicinal preparation.

2. The diffuser according to claim 1, wherein:
the frame comprises a wall having a first side and a second side;
the heat source disposed on the first side of the wall; and
the plurality of strips disposed on the second side of the wall.

3. The diffuser according to claim 2, comprising a plurality of brackets attached to the second side of the wall, the brackets and the second side of the wall defining a plurality of slots, the plurality of strips removably disposed in the plurality of slots.

4. The diffuser according to claim 3, comprising a plurality of refills, each refill including one of the plurality of strips and a refill frame, the one of the plurality of strips of heat-responsive material disposed on the refill frame.

5. The diffuser according to claim 4, wherein the wall is cylindrical in shape.

6. The diffuser according to claim 1, comprising:
a plurality of sockets attached to the frame; and
a plurality of refills, each refill including one of the plurality of strips and a refill frame, the one of the plurality of strips of heat-responsive material disposed on the refill frame,
at least a part of each of the plurality of refills disposed in one of the plurality of sockets.

7. The diffuser according to claim 6, wherein the refill frame defines an opening and the one of the plurality of strips of heat-responsive material is disposed in the opening.

8. The diffuser according to claim 1, wherein the frame defines a wall having a plurality of openings defined therein, comprising:
a plurality of refills, each refill including one of the plurality of strips, a refill frame, and a fastener, the one of the plurality of strips of heat-responsive material disposed on the refill frame and the fastener disposed through one of the plurality of openings in the wall of the frame to attach the refill to the frame.

9. The diffuser according to claim 8, wherein the refill frame defines an opening and the one of the plurality of strips of heat-responsive material is disposed in the opening.

10. The diffuser according to claim 1, wherein at least one of the plurality of strips includes a different active component than the other strips.

11. The diffuser according to claim 1, wherein at least one of the plurality of strips includes a fragrance that is different than that of the other strips.

12. The diffuser according to claim 1, wherein at least one of the plurality of strips includes a fragrance and at least another of the plurality of strips includes an insecticide or a medicinal preparation.

13. A diffuser comprising:
a frame including a receptacle;
a candle disposed in the receptacle;
a plurality of refills attached to the frame and disposed about the receptacle, each of the plurality of refills including a layer of heat-responsive material, the heat-responsive material comprising a carrier and an active component, the active component comprising at least one of a fragrance, an insecticide, or a medicinal preparation.

14. The diffuser according to claim 13, wherein the frame comprises a wall with a first side and a second side with a plurality of slots, the candle disposed on the first side of the wall and the plurality of refills disposed in the plurality of slots.

15. The diffuser according to claim 13, comprising a plurality of sockets attached to the frame, at least a part of each of the plurality of refills disposed in one of the plurality of sockets.

16. The diffuser according to claim 13, wherein:
the frame defines a wall having a plurality of openings defined therein; and
each of the plurality of refills includes a fastener, the fastener being disposed through one of the plurality of openings in the wall of the frame to attach the refill to the frame.

17. The diffuser according to claim 13, wherein the refill frame defines an opening and the layer of heat-responsive material is disposed in the opening.

18. The diffuser according to claim 13, wherein the layer of heat-responsive material of at least one of the plurality of refills includes a different active component than the layer of heat-responsive material of the other refills.

19. The diffuser according to claim 13, wherein the layer of heat-responsive material of at least one of the plurality of refills includes a fragrance that is different than that of the layers of heat-responsive material of the other refills.

20. The diffuser according to claim 13, wherein the layer of heat-responsive material of at least one of the plurality of refills includes a fragrance and the layer of heat-responsive material of at least another of the refills includes an insecticide or a medicinal preparation.
